(19) United States
(12) Patent Application Publication
(10) Pub. No.: US 2026/0191557 A1
(43) Pub. Date: Jul. 9, 2026

(54) SYSTEMS AND METHODS FOR TREATING SCARS

(71) Applicant: Behrooz Barikbin, North York (CA)

(72) Inventor: Behrooz Barikbin, North York (CA)

(21) Appl. No.: 19/282,662

(22) Filed: Jul. 28, 2025

Related U.S. Application Data
(60) Provisional application No. 63/743,173, filed on Jan. 8, 2025.

(51) Int. Cl. A61B 17/3209; A61B 17/00; A61B 17/32; A61B 18/00; A61B 18/04; A61B 18/20; A61L 24/00; A61L 24/08; A61L 27/36

(52) U.S. Cl. CPC A61B 17/32093; A61B 18/042; A61B 18/203; A61L 24/0015; A61L 24/08; A61L 27/3604; A61B 2017/00792; A61B 2017/320044; A61B 2018/00327; A61L 2300/402; A61L 2300/43; A61L 2430/34

(57) ABSTRACT

A minimally invasive method of treating nasal scars and deformities caused by rhinoplasty, burns, or trauma comprising (1) injecting a personalized tumescent solution into the nasal scar tissue; (2) performing subcision on the scarred area using a blunt cannula (BBSB TYPE D) to release fibrotic bands and adhesions; (3) treating the scarred area with fractional CO2 laser or plasma (Plexr®) for scar resurfacing; and (4) injecting micro-fat and nano-fat into the scarred area about two weeks later to rejuvenate the skin and improve healing and vasculature. This approach allows for better surgical outcomes in revision rhinoplasty or eliminates the need for revision surgery in certain cases.

### CROSS-REFERENCE TO RELATED APPLICATIONS

This U.S. Non-Provisional Patent Application claims the benefit of U.S. Provisional Application No. 63/743,173 filed on Jan. 8, 2025, titled “SYSTEMS AND METHODS FOR TREATING SCARS”, by inventor Behrooz Barikbin, the contents of which are expressly incorporated herein by this reference as though set forth in their entirety.

### FIELD OF THE DISCLOSURE

The present disclosure, in general, relates to systems and methods for treating scars. More specifically, the present disclosure relates to a minimally invasive method of treating nasal scars and deformities caused by rhinoplasty, burns, or trauma.

### BACKGROUND

In 2016, inventor Behrooz Barikbin co-authored an article in the American Society for Dermatologic Surgery titled Blunt Blade Subcision: An Evolution in the Treatment of Atrophic Acne Scars (published by Published by Wolters Kluwer Health, Inc.) (ISSN: 1076-0512; Dermatol Surg 2016; 0:1-7*DOI: 10.1097/DSS.0000000000000650) (the “2016 Article”). The objective of the 2016 Article, the contents of which are hereby incorporated herein by this reference as though set forth in their entirety, was to improve the efficacy of the subcision procedure using a newly designed blunt subcision blade. The use of the specific blunt subcision blade, or Barikbin's subcision blade, was found to be effective and safer than using a sharp subcision blade. Specifically, there was a moderate to marked improvement of atrophic scars in 15 cases out of 18 (83.3%).

5% to 15% of rhinoplasty (nose or nasal surgery) results in postoperative deformities, which often require revision surgery. Rhinoplasty complications can include atrophy, cyst formation, numbness, adhesions, and fibrosis, which is the thickening of connective tissue, which can restrict movement and function. Unpredictable scarring and contraction during the post rhinoplasty healing process can cause the soft tissue to adhere (adhesions) to the nasal dorsum, especially at critical junctions like the keystone area and the tip/lower lateral cura. The chances of having fibrosis and/or adhesions can be increased by a variety of factors, including extensive surgical history, genetics, and comorbidities, such as diabetes, thyroid disorders, and connective tissue diseases.

Current techniques to prevent long term skin damage and shrinkage are limited, emphasizing the need for meticulous surgical techniques that preserve soft tissue and blood vessels. In some cases, the nasal deformities due to rhinoplasty (or due to trauma or burns), are so severe that revision surgery is exceedingly difficult or even impossible. Having developed the blunt subcision blade and having had success in treating acne scars, which are typically on the cheek and jaw portions of the face, inventor Behrooz Barikbin was inspired to use the blunt subcision blade to repair the nose so that revision surgery could be performed or performed more easily.

Prior to the present disclosure, there has not been a satisfactory solution for addressing the recalcitrant problem of treating nasal deformities so severe that revision surgery was not an option.

### SUMMARY

To minimize the limitations in the prior art, and to minimize other limitations that will become apparent upon reading and understanding the present specification, the present disclosure discloses new and useful scar treatment devices, systems, and methods.

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented hereinbelow. It is to be understood that both the following general description and the following detailed description are exemplary and explanatory only and are not restrictive.

One embodiment is a minimally invasive method of treating nasal scars and deformities caused by rhinoplasty, burns, or trauma, and may comprise the following four main steps:
1. Injection of Tumescent Solution (that may contain non cross link hyaluronic acid, lidocaine, epinephrine, and normal saline) and may be injected into the affected area (usually 5 to 10 cc is enough). This solution aids in anesthesia, reduces bleeding via vasoconstriction, and allows hydro-dissection, which separates fibrotic tissue layers.
2. Blunt Subcision. Subcision is performed using a specialized blunt cannula designed by Dr. Behrooz Barikbin. This step effectively releases fibrotic bands and adhesions without damaging nerves or blood vessels. The precision of the cannula ensures minimal trauma.
3. Laser or Plasma Therapy: Fractional CO2 laser and or plasma technology (e.g., Plexr®) is applied immediately after subcision. This step smooths the external appearance of scars and enhances the overall treatment effectiveness by targeting surface irregularities.
4. Fat Injection (Micro-fat and Nano-fat). Two weeks later, micro-fat and nano-fat grafting are performed to improve vascularity and scar texture. These steps leverage the regenerative properties of adipose stem cells, enhancing healing and achieving aesthetic and functional improvements.

The present disclosure may be a method that combines blunt blade subcision, plasma dermabrasion (such as Plexr®) and/or fractional carbon dioxide (CO2) laser therapy, and fat injection. This unique combination provides the healthcare provider with the ability to release scar tissue without damaging nerves or vessels, ensuring improved healing and appearance. This combination has previously never been used to treat patients who suffer from more compact scars on the nose, which cannot be corrected through revision rhinoplasty due to the complexity of the cases for surgeons. The method of the present disclosure precisely releases scar tissue without causing nerve or vascular damage, and the fat injection enhances vascularity with the help of fat stem cells, acting as an autologous filler. This ensures better healing and surgical outcomes.

The method of the present disclosure restores the confidence of patients and, in some cases, avoids unnecessary plastic surgeries. Further it is a cost-effective solution for severe nasal scars, for which insurance will very frequently not pay. It not only helps plastic surgeons achieve more desirable results but also brings hope to patients. In some cases, reopening the tissues is impossible, but the method of the present disclosure brings hope even for those infrequent cases.

The methods, systems, and devices of the present disclosure cause the release of tissue adhesions caused by severe scars and significantly improves the quality and appearance of these scars. The methods, systems, and devices of the present disclosure effectively improve severe scars, both internally and externally, especially on the nose, by using a combination of laser treatments, injectables, and subcision.

The method of the present disclosure may be a new, useful, and not obvious minimally invasive method for treating stubborn nasal scars caused by surgery, trauma, or burns. The method is especially useful when further surgery is difficult or impossible.

The method preferably involves four key steps.
1) A novel liquid mixture (tumescent solution) is injected into the scarred area of the nose, which is being prepared to be subscised, to numb the tissue, reduce bleeding, and gently separate scarred layers. This is sometimes referred to herein as “hydrodissection.” Typically, 5-10 cc of the tumescent solution is enough for making the scarred area full of this novel fluid (tumescent solution. Preferably, the injection of the liquid is done quickly and all at once, which provides the proper pressure for helping releasing fibrotic tissues at this first stage as much as possible.
2) Preferably a unique blunt blade (designed by Dr. Barikbin) is inserted under the skin to safely break up the thick, tight scar bands and release fibrotic tissue without cutting nerves or blood vessels.
3) A laser, such as a fractional CO2 laser, Erbium Yag laser, or plasma jet device, may be used to smooth and improve the appearance of the scar on the skin.
4) About two weeks later, fat is preferably injected into the treated area to further heal the area and fill/soften the skin using the body's own fat and stem cells. In this stage it is preferred that about 1 cc of micro-fat is injected under the depressed area of the scar and about 2 cc nano-fat is injected inside the derm, hypoderm and the scar itself.

The tumescent solution preferably contains lidocaine for numbing, epinephrine to reduce bleeding, hyaluronic acid to hydrate and stimulate healing, and saline for separating the tissue.

The blunt blade used in the procedure is preferably selected for its specific size and shape to make it safer and more effective than sharp tools that have been used previously. The entrance point of subcision blade is preferably made by a G-21 (green) hypodermic needle, which is so small that no entrance scar is created.

Laser or plasma jet (such as Plexr®) treatment is preferably done immediately after the subcision procedure on the scar tissue because the skin is already numb and the healing time of laser or plasma jet therapy will merge with the healing time of the subcision step, which reduces total downtime of the procedure. Frequently, plasma therapy and fractional CO2 laser therapy may both be done immediately after the subcision step. When this is done the results may be enhanced because results show that there is a synergistic effect in making the final look of the scar better.

Fat is preferably injected two weeks later for long-term healing, improves the scar's appearance significantly, and because there are stem cells in the fat. The stem cells have growth factors that help with vasculature and texture of the area, which is very useful in making the area suitable for a surgeon to perform revision surgery, if that is even necessary after the procedure of the present disclosure.

In general, the method of the present disclosure helps prepare the nose for future plastic surgery—or even makes surgery unnecessary—by improving blood flow, skin quality, and scar texture.

One embodiment may be a method of treating scars comprising: providing a tumescent solution; injecting the tumescent solution into a scarred area of a nose of a patient; performing subcision on the scarred area; treating the scarred area of the nose with one or more of a laser treatment and plasma treatment; and injecting fat into the scarred area of the nose. The tumescent solution may comprise hyaluronic acid, lidocaine, epinephrine, and normal saline. Preferably, between 5 to 10 cc of the tumescent solution may be injected. The tumescent solution aids in anesthesia, reduces bleeding via vasoconstriction, and provides for hydro-dissection of the scarred area. The tumescent solution may be 20 mL and comprise 3 mL lidocaine 2%, 0.02 mL epinephrine 1:1000, 3 mL non-crosslinked hyaluronic acid 2%, and 13.98 mL normal saline. The subcision may be performed using a blunt subcision blade. The blunt blade of the blunt subcision blade may be 1.5 mm to 2.2 mm in width and 50 mm to 70 mm in length, and wherein the blunt blade has a tapered non-sharp blunt tip and dual flat lateral edges. A plurality of fibrotic bands and adhesions are released by the blunt subcision blade without damaging nerves or blood vessels. The laser treatment may be fractional CO2 laser treatment. The plasma treatment may be Plexr® plasma treatment. The laser treatment and plasma treatment may be performed immediately after the subcision step. The laser treatment and plasma treatment smooths an external appearance of the scar area. The injecting fat into the scar area step may be performed approximately 2 weeks after the laser or plasma treatment step. The injecting fat into the scar area step may comprise injecting a micro-fat and a nano-fat into the scar area. The injecting fat into the scar area step uses adipose stem cells, which have regenerative properties, which promotes healing of the scar area.

Another embodiment may be a method of treating scars comprising: providing a tumescent solution; injecting the tumescent solution into a scarred area of a nose of a patient; performing subcision on the scarred area; treating the scarred area of the nose with one or more of a laser treatment and a plasma treatment; and injecting fat into the scarred area of the nose; wherein the tumescent solution comprises hyaluronic acid, lidocaine, epinephrine, and normal saline; wherein the subcision may be performed using a blunt subcision blade; wherein a plurality of fibrotic bands and adhesions are released by the blunt subcision blade without damaging nerves or blood vessels; wherein the laser treatment and the plasma treatment may be performed immediately after the subcision step; wherein the laser treatment and the plasma treatment smooths an external appearance of the scar area; wherein the injecting fat into the scar area step may be performed approximately 2 weeks after the laser treatment and plasma treatment; wherein the injecting fat into the scar area step comprises injecting a micro-fat and a nano-fat into the scar area; and wherein the injecting fat into the scar area step uses adipose stem cells, which have regenerative properties, which promotes healing of the scar area.

Another embodiment may be a method of treating scars comprising: identifying a scarred area on a nose of a patient; performing subcision on the scarred area; wherein the subcision may be performed using a blunt subcision blade; wherein a plurality of fibrotic bands and adhesions are released by the blunt subcision blade without damaging nerves or blood vessels; a blunt blade of the blunt subcision blade may be 1.5 mm to 2.2 mm in width and 50 mm to 70 mm in length, and wherein the blunt blade has a tapered non-sharp blunt tip and dual flat lateral edges. The method may further comprise: providing a tumescent solution; injecting the tumescent solution into the scarred area of the nose of the patient; treating the scarred area of the nose with one or more of a laser treatment and a plasma treatment; and injecting fat into the scarred area of the nose. The tumescent solution of 20 mL comprises 3 mL lidocaine 2%, 0.02 mL epinephrine 1:1000, 3 mL non-crosslinked hyaluronic acid 2%, and 13.98 mL normal saline. A plurality of fibrotic bands and adhesions are released by the blunt subcision blade without damaging nerves or blood vessels. One or more of a laser and plasma treatment may be performed immediately after the subcision step. The laser and plasma treatment step smooths an external appearance of the scar area. The injecting fat into the scar area step may be performed approximately 2 weeks after the laser treatment and plasma treatment. The injecting fat into the scar area step may comprise injecting a micro-fat and a nano-fat into the scar area; and the injecting fat into the scar area step may use adipose stem cells.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure. As it will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is an illustration of five embodiments of a blunt subcision blade.

FIG. 2A is an illustration of five embodiments of the blunt subcision blade.

FIG. 2b is an illustration of one embodiment of the blunt subcision blade.

FIG. 3 is an illustration showing the injection of the tumescent solution.

FIG. 4 is an illustration showing the fibrosis tissue and the tumescent solution.

FIG. 5 is an illustration showing Hydro-expansion and Hydro-dissection (degradation) of the fibrosis due to tumescent solution.

FIG. 6 is an illustration showing injection of tumescent solution into a scar.

FIG. 7 is an illustration showing constriction of blood vessels due to the injection of the tumescent solution.

FIG. 8 is an illustration showing injection of tumescent solution and also showing the fibrosis, which is shown causing indentations and adhesions, which cause scars.

FIG. 9 is an illustration showing subcision being performed with Barikbin's blunt blade.

FIG. 10 is an illustration showing subcision from the same entry point, different angle with Barikbin's blunt blade.

FIG. 11 is an illustration showing subcision with Barikbin's blunt blade, new entry point and different angle.

FIG. 12 is an illustration showing the blunt subcision blade/tool being used to disrupt the fibrotic tissue.

FIG. 13 is an illustration showing subcision (breaking up of fibrosis tissue) with Barikbin's blunt blade tool.

FIG. 14 is an illustration showing that the subcision step has lifted the indentations and created space within the dense fibrotic bands, preparing the area for fat injection in the following weeks.

FIG. 15 is an illustration showing that the subcision step has lifted the indentations and created space within the dense fibrotic bands, preparing the area for fat injection in the following weeks.

FIG. 16 is an illustration showing laser therapy. To further enhance results, fractional CO2 laser therapy may be applied immediately after (or concurrently with) subcision.

FIG. 17 is an illustration showing laser therapy may provide optimal skin rejuvenation.

FIG. 18 is an illustration showing an injection needle with fat.

FIG. 19 is an illustration showing fat/micro-fat being injected.

FIG. 20 is an illustration showing fat/micro fat having been injected.

FIG. 21 is an illustration showing additional fat/micro fat being injected deep beneath the skin to fill depressed areas beneath the scar.

FIG. 22 is an illustration showing nano-fat is applied intradermally to rejuvenate the skin surface and refine scar texture.

FIG. 23 is an illustration showing additional nano-fat being injected just under the top layer of skin.

FIG. 24 is an illustration showing before and after (left and right respectively) treatment via the method of the present disclosure, which has been so successful that further revision surgery is unnecessary.

FIGS. 25A-D are illustrations of images showing positive results from the method of the present disclosure.

FIGS. 26A-D are illustrations of a patient treated with the method of the present disclosure.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers, or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 0.001-10% from the indicated number or range of numbers.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

As used herein, the term “Barikbin's subcision blade”, “Barikbin's blunt blade(s)”, “Barikbin's blunt blade tool(s)”, “blunt subcision blade”, “blunt blade tool”, and “blunt tissue separator”, all refer to a subcision blade that has a blunt tip and edges (sides). Preferably, the blunt subcision blade used for the method of the current disclosure may be a stainless-steel blade in varied sizes. Typically, a more compact blade is used for subcision on the nose. The blunt edges may gradually taper to form two flat surfaces (similar to a standard screwdriver (non-Phillips head)). However, the tool preferably remains blunt and noncutting (not sharp) at the tip. The edges may be constructed as narrowly as possible to enable the release of fibrous strands using only light sheering force. Preferably, the blunt subcision blade is configured to prevent blood vessel laceration and nerve trauma during subdermal maneuvering. The blade length preferably allows distant scars in the area to be reached through a single penetration site. The blade handle may be ergonomic and may preferably have a thumb depression on one side to ensure the blade is held correctly, with the flat surfaces being horizontal and the blunt edges lateral.

The first step of the method is providing and injecting a tumescent solution to the affected area. Preferably, the tumescent solution is comprised of lidocaine, epinephrine, and normal saline. The lidocaine in the tumescent solution facilitates the process of anesthesia. The addition of epinephrine to the tumescent solution causes vasoconstriction, effectively reducing blood loss during procedures. This is known as epinephrine-induced vasoconstriction. The use of sterile fluids (normal saline) provides for hydro-dissection, which is a valuable technique that involves the injection of sterile fluids for hydraulic elevation and separation of tissues in a desired surgical plane. Further, the hydrostatic pressure of the tumescent fluid helps to minimize bleeding by compressing the tissue and blood vessels.

Step two of the method is blunt blade subcision, sometimes referred to as Barikbin's blunt blade subcision, which releases fibrotic bands and tears anchoring fibers. As such, the skin surface is freed from the tethering forces produced by these bands and prepares the tissue for improved healing and remodeling.

Previously, subcision was performed with a sharp blade, needle, or cannula. Such sharp instruments result in poorer results and pose significant risks of nerve and vascular damage. Specifically, devices like the Nokor™ needle are not useful in nasal scar tissue separation because the hematoma created by sharp subcision can lead to new fibrotic masses, creating additional problems for the surgeon.

The blunt blade tool of the present disclosure, which may be referred to as a tissue separator, preferably may have a specific range of sharpness/bluntness and flexibility to effectively release fibrotic bands without causing excessive damage to surrounding tissue. The handle should be easy to grip without slipping and should allow the operator to perform subcision from several aspects (points or locations) of the scar.

The subcision tool preferably should be made of medical-grade stainless-steel to ensure sterility, durability, and autoclavability (for repeated use).

The use of a blunt blade allows for safe tearing of fibrotic and anchoring fibers without inducing bleeding or hematoma, effectively releasing the skin from underlying stiffness.

Steps three and four may preferably be fractional CO2 Laser and/or plasma treatment, fat injection, microneedling, and/or fractional radio frequency energy treatment (typically delivered by microneedling.

The subcision precisely releases scar tissue without causing nerve or vascular damage, and the fat injection enhances vascularity with the help of fat stem cells, acting as an autologous filler. This ensures better healing and surgical outcomes.

The plasma (Plexr®) treatment and/or fractional CO2 laser treatment improves the appearance of the scars and should preferably be done concurrently with the subcision. Fractional CO2 laser treatment smooths and improves the external appearance of the scars.

Preferably, the laser should operate within a specified wavelength (commonly around 10,600 nm) and power range to ensure optimal resurfacing and minimal collateral damage. Plasma treatments, such as Plexr® may be beneficial for very fine treatment of scars.

Autologous micro-fat injections act as a filler and nano-fat improves the quality of the scar surface. Both enhance vascularization and the condition of the area using the regenerative properties of fat stem cells.

Preferably, the micro-fat may be injected using a 22G cannula, and nano-fat may be injected using meso needles of gauge 30 to ensure precise placement and minimal trauma to the tissue. Meso, or middle, allows the fat to be injected into the middle of the skin layer. These parameters are preferable to maintain the effectiveness and safety of the procedure, ensuring that each component performs its intended function without causing adverse effects.

Preferably both laser and fat injection treatments are both performed because together they ensure both functional and aesthetic improvements.

Although it is possible for soft-tissue or synthetic fillers to replace fat injections, soft-tissue or synthetic fillers lack regenerative benefits. Additionally, synthetic fillers carry the risk of foreign body reactions and could potentially worsen fibrosis, making the condition more complicated to treat. And, while alternative resurfacing methods, such as dermabrasion or microneedling, could be used in place of laser or plasma therapy, they may be less effective.

In some cases, fat injections may be replaced (or partially replaced by platelet rich plasma therapy or stem cell injections.

Combining subcision with plasma and/or laser therapy in the same session has both a synergistic effect and also merges the recovery time for each procedure, enhancing the overall efficacy and efficiency.

It is preferred to do the subcision and laser treatments in the same session and then complete the fat injection about two weeks later, because this gives the treatment area time to be emptied of the tumescent solution. Eliminating fat injections would result in poorer vascularization and healing outcomes. Eliminating the laser step would save cost but lead to suboptimal external scar appearance. In some embodiments, advanced imaging techniques such as ultrasound or optical coherence tomography may be used to further enhance the precision and safety of subcision. In some embodiments, the fat injections may incorporate growth factors and/or platelet rich plasma to further improve tissue regeneration and healing.

Like all health and surgical methods, the method of the present disclosure is subject to success based on the status of the patient. Patients with severely compromised vascular health or systemic conditions that prevent proper wound healing may have unfavorable outcomes. Additionally, patients without significant fibrotic bands would not benefit significantly from subcision, although in these cases, laser therapy and fat injection might still be helpful. Additionally, for depressed scars caused by destroyed cartilage, the method of the present disclosure might have limited benefit.

The method of the present disclosure is preferably performed after an initial rhinoplasty, trauma, or burn, and several months before revision rhinoplasty.

The method aims to provide a solution for recalcitrant nasal scars that complicate revision rhinoplasty. The procedure preferably includes: 1. Injection of a personalized Tumescent Solution. The solution may comprise lidocaine for local anesthesia, epinephrine for vasoconstriction, Hyaluronic acid for making the solution more viscose and increase healing properties and normal saline for hydro-dissection. This step reduces bleeding and prepares the fibrotic tissue for safe subcision. 2. Blunt Subcision with Barikbin's Cannula Using a blunt-tipped, noncutting cannula type D, fibrotic bands are released safely. Unlike sharp subcision tools, this cannula minimizes risks of nerve or vascular damage. Subcision lifts tethered skin, creating a more uniform surface. 3. Simultaneous Laser or Plasma Treatment. To enhance results, fractional CO2 laser therapy or Plexr® plasma treatment is applied during the same session. This dual approach treats deeper tissue with subcision and refines surface scars, optimizing outcomes. 4. Fat Injection (Micro-fat and Nano-fat) After a two-week healing period, micro-fat is injected to restore volume, and nano-fat is applied intradermally to refine texture and improve vascularity. These steps utilize the regenerative properties of fat stem cells for long-term benefits. The method prepares nasal tissues for easier and safer revision surgery by improving skin quality, reducing adhesions, and enhancing blood supply. In some cases, it eliminates the need for surgery altogether.

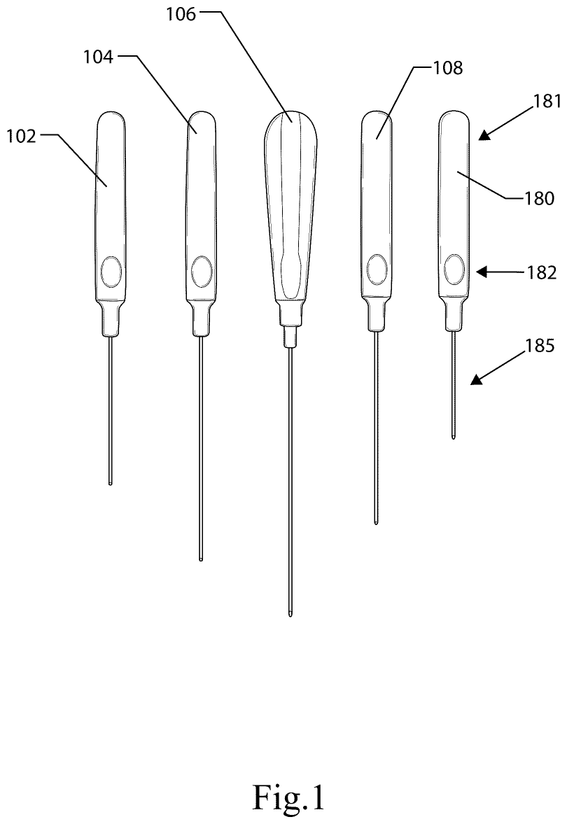

FIG. 1 is an illustration of five embodiments of a blunt subcision blade. As shown in FIG. 1, the blunt subcision blades 102, 104, 106, 108, 180, may have various handle shapes and lengths. Blunt subcision blade 180 shows a preferred embodiment of the tool that is used for breaking up fibrosis tissue in the nose. Blunt subcision blade 180 is shown with a blunt blade 185, easy to grip handle 181, and thumb depression 182, to prevent slippage and to ensure that the tool is held at the right angle for breaking up fibroses.

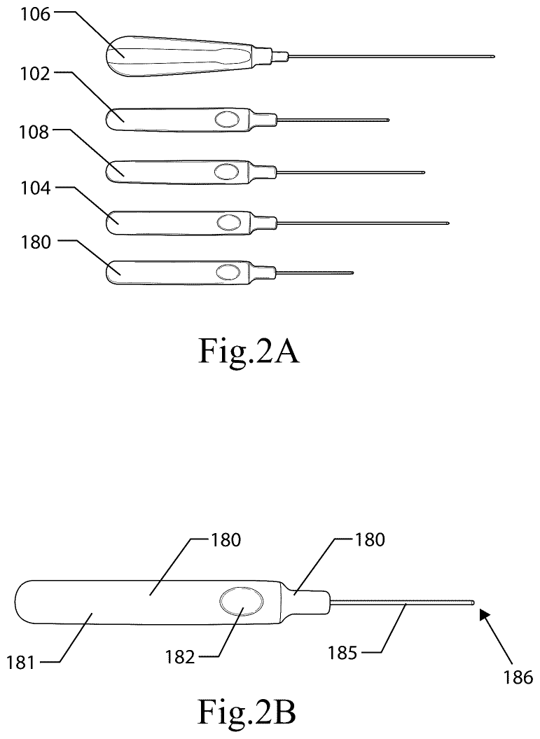

FIG. 2A is an illustration of five embodiments of the blunt subcision blade. D is the preferred blade for nose subcision procedures. FIG. 2A shows the relative sizes of the blunt subcision blades 102, 104, 106, 108, 180, shown on the backdrop of a standard piece of graph paper.

FIG. 2B is an illustration of one embodiment of the blunt subcision blade. Blunt subcision blade 180 shows a preferred embodiment of the tool that is used for breaking up fibrosis tissue in the nose. Blunt subcision blade 180 is shown with a blunt blade 185, easy to grip handle 181, and thumb depression 182, to prevent slippage and to ensure that the tool is held at the right angle for breaking up fibroses. The blade 185 is shown as cylindrical at the base and tapers in a flat manner on two opposite sides that ends in blunt tip 186. Preferably, blunt subcision blade 185 is approximately 1.5 mm to 2.2 mm in width, 50 mm to 12, 70 mm in length, and has a tapered noncutting (non-sharp) blunt tip with dual flat lateral edges, designed for horizontal fan-like movement. The preferred embodiment for subcision of nasal scars may have an ergonomic handle.

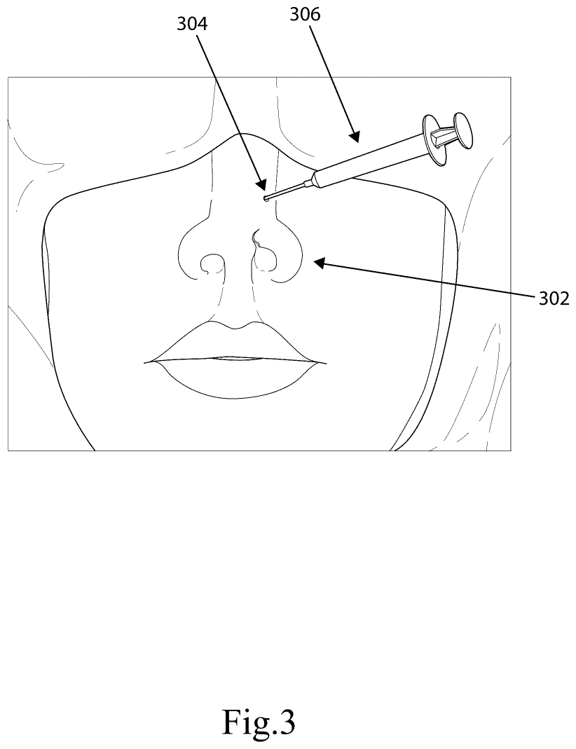

FIG. 3 is an illustration showing the injection of the tumescent solution. As shown in FIG. 3, the first step in the method of the present disclosure for treating nasal deformities is to inject the scar area 302 with a tumescent solution using hypodermic needle 306. As shown in FIG. 3 the needle may penetrate the skin at injection site 304. In one embodiment, the tumescent solution may preferably include non-crosslink hyaluronic acid, lidocaine, epinephrine, and normal saline. Typically, 5 to 10 cc of the solution is enough. The solution aids in anesthesia, reduces bleeding via vasoconstriction, and allows hydro-dissection, which separates fibrotic tissue layers.

The lidocaine in the tumescent solution facilitates the process of anesthesia. The addition of epinephrine to the tumescent solution causes vasoconstriction, effectively reducing blood loss during procedures. This is known as epinephrine-induced vasoconstriction. The use of sterile fluids (normal saline) provides for hydro-dissection, which is a valuable technique that involves the injection of sterile fluids for hydraulic elevation and separation of tissues in a desired surgical plane. Further, the hydrostatic pressure of the tumescent fluid helps to minimize bleeding by compressing the tissue and blood vessels. The non-cross-linked hyaluronic acid (HA) rejuvenates the skin and acts as a bio-revitalization treatment. HA is a glycosaminoglycan that is naturally found in the extracellular matrix of the skin, and it has several beneficial effects on the skin such as hydration and stimulation. In another embodiment, the tumescent solution may comprise lidocaine, epinephrine, and normal saline.

Normal saline may preferably be a sterile, clear solution containing approximately 0.9% sodium chloride (NaCl) in water.

In one embodiment, the tumescent solution comprises approximately: 3 mL of lidocaine 2% solution (equivalent to 60 mg); 0.02 mL of epinephrine 1:1000 (equivalent to 20 micrograms, resulting in a final dilution of 1:100,000); 3 mL of non-crosslinked hyaluronic acid 2% solution (equivalent to 60 mg, providing a final concentration of 0.3%); and 13.98 mL of normal saline (0.9% NaCl), to make a total volume of 20 mL for injection into the scar area. This formulation ensures enhanced anesthesia, sufficient vasoconstriction, improved hydro-dissection, and superior bio-revitalization due to the higher hyaluronic acid content. This tumescent solution is particularly suitable for releasing scarred and fibrotic nasal tissue.

In another preferred embodiment, approximately 5 to 10 cc of the tumescent solution is injected into the scarred nasal area, preferably using a 29G or 30G hypodermic needle, which allows for rapid, high-pressure, and highly precise hydro-expansion. In some cases, particularly in very fibrotic or delicate regions, a 25G blunt cannula may be used as an alternative for safer, more controlled infiltration of the injected tumescent solution. Preferably, the tumescent solution is delivered quickly and forcefully to mechanically separate fibrotic tissue planes (hydrodissection), thereby loosening adhesions even before manual subcision begins. The tumescent solution may comprise 3 mL lidocaine 2% (60 mg), 0.02 mL epinephrine 1:1000(20 μg), 3 mL non-crosslinked hyaluronic acid 2% (60 mg), and 13.98 mL of 0.9% normal saline, totaling 20 mL. This combination provides anesthesia, vasoconstriction, tissue distention, and regenerative stimulation, optimizing the treatment zone for the subsequent steps.

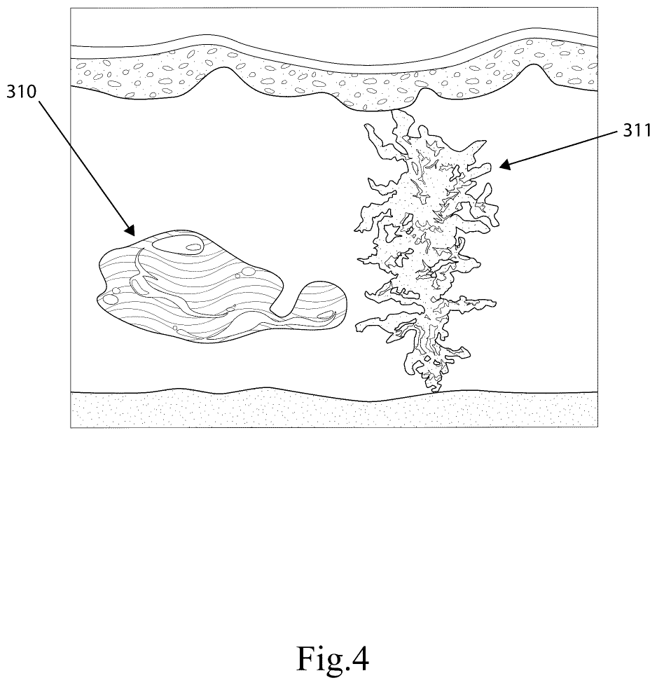

FIG. 4 is an illustration showing the fibrosis tissue and the tumescent solution. As shown in FIG. 4, the tumescent solution 310 and may be injected into the affected area of the skin and may come into contact with fibrosis tissue 311.

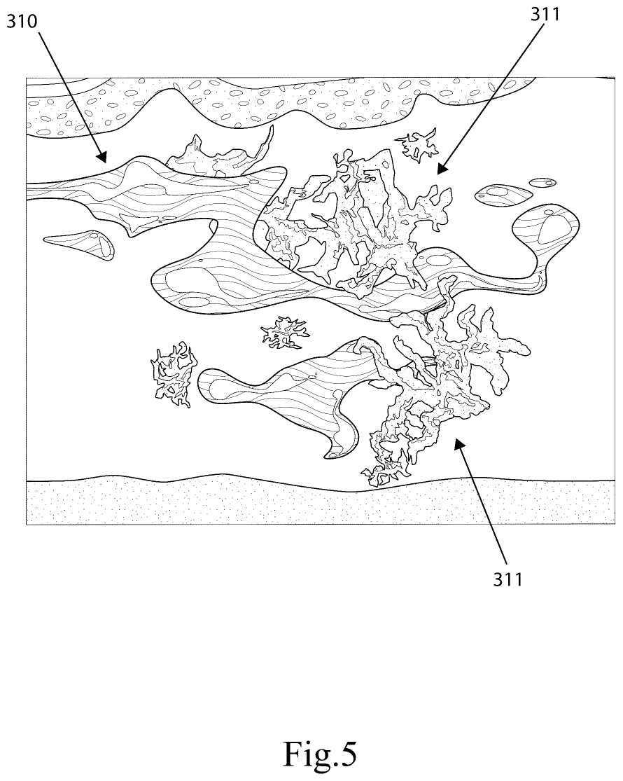

FIG. 5 is an illustration showing Hydro-expansion and Hydro-dissection (degradation) of the fibrosis due to tumescent solution. FIG. 5 shows that tumescent solution 310 is breaking apart or degrading fibrosis 311. FIG. 5 also shows the hydro-expansion effect of the solution.

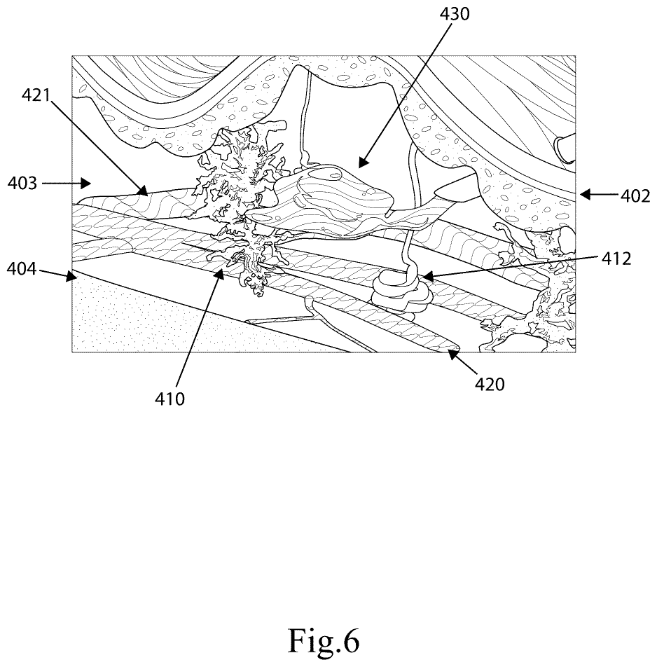

FIG. 6 is an illustration showing injection of tumescent solution into a scar. FIG. 6 shows that the scar area is on the skin, which is comprised of epidermis 402, dermis 403, which supports sweat glands 412, and hypodermis 404, which supports blood vessels 420, 421. As shown, dermis 403 of the scar area has multiple fibrotic masses 410 that are connected to epidermis 402 and hypodermis 404, which causes the shown indentations and adhesions. FIG. 6 also shows that epidermis 402 may comprise various strata, such as stratum corneum, lucideum, granulosum, spinosum, and basale. FIG. 6 also shows tumescent solution 430 being injected into dermis 403.

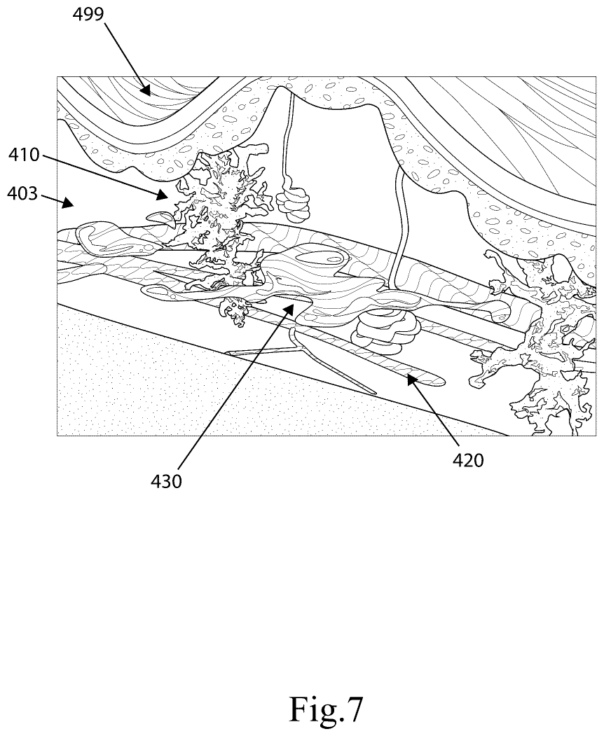

FIG. 7 is an illustration showing constriction of blood vessels due to the injection of the tumescent solution. FIG. 7 shows that tumescent solution 430, which was injected into dermis 403 causes constriction in blood vessels 420, due to epinephrine. FIG. 7 also shows indentation 499 caused by fibrosis 410.

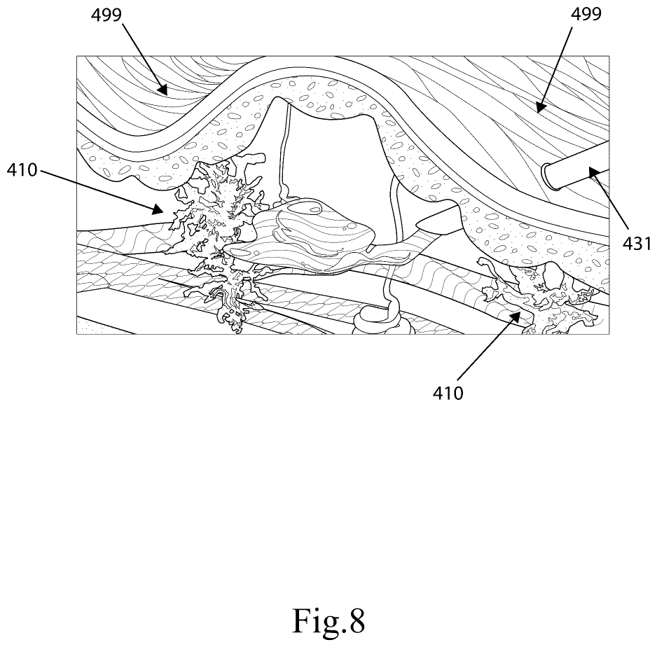

FIG. 8 is an illustration showing injection of tumescent solution and also showing the fibrosis, which is shown causing indentations and adhesions, which cause scars. Fibrosis 410 is shown causing indentations 499, which contribute to the scaring and deformities of the none. FIG. 8 also shows the tumescent solution injection needle 431 delivering the solution hypodermically.

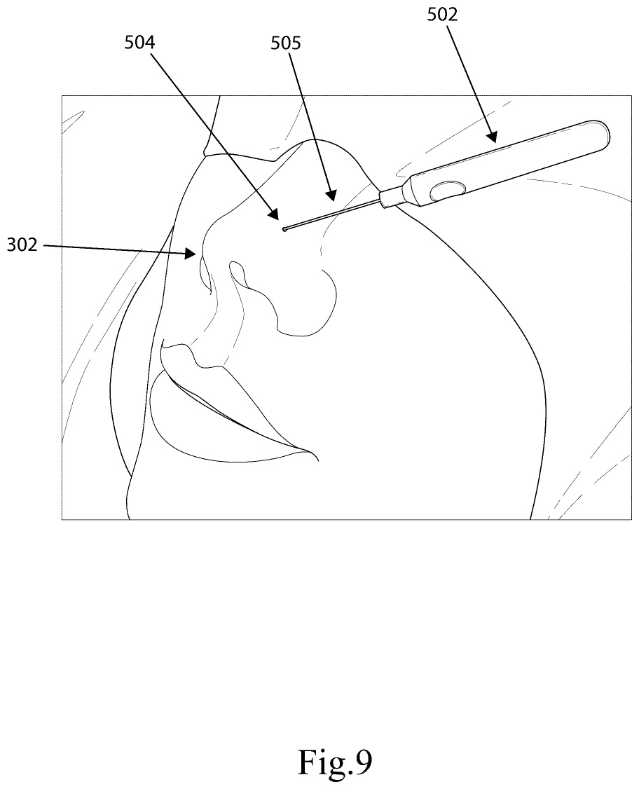

FIG. 9 is an illustration showing subcision being performed with Barikbin's blunt blade. As shown in FIG. 9, the second step in the method of the present disclosure for treating nasal deformities is to use blunt subcision blade 502 to penetrate/enter penetration point 504 the skin at scar area 302. Scar area 302 is shown with a nasal deformity that may have been caused by prior nasal surgery (rhinoplasty) or trauma. The blunt blade 505 of blunt subcision blade 502, enters the skin at penetration point 504 and is moved back in forth as maneuvered by a surgeon to break up fibrotic tissue (sometimes referred to as subcision) without damaging blood vessels or nerves, or causing further scaring.

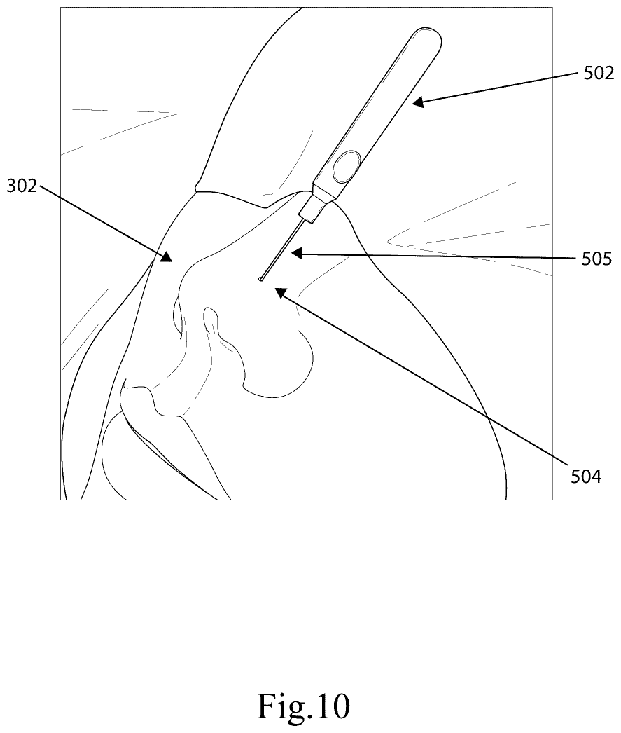

FIG. 10 is an illustration showing subcision from the same entry point, different angle with Barikbin's blunt blade. As shown in FIG. 10, the blunt blade 505 of blunt subcision blade 502, enters the skin at penetration point 504 at a different angle and may be moved back in forth as maneuvered by a surgeon to further break up fibrotic tissue. The blunt subcision blade 502 may penetrate the skin in different entry points, as determined by the surgeon, at or near the scar area 302.

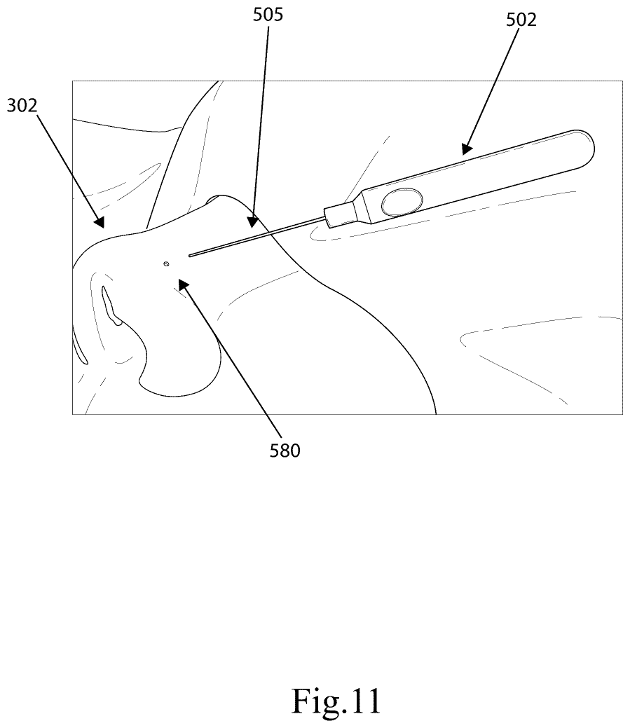

FIG. 11 is an illustration showing subcision with Barikbin's blunt blade, new entry point and different angle. As shown in FIG. 11, the blunt blade 505 of blunt subcision blade 502, enters the skin at a different penetration point 580 and may be moved back in forth as maneuvered by a surgeon to further break up fibrotic tissue in scar area 302.

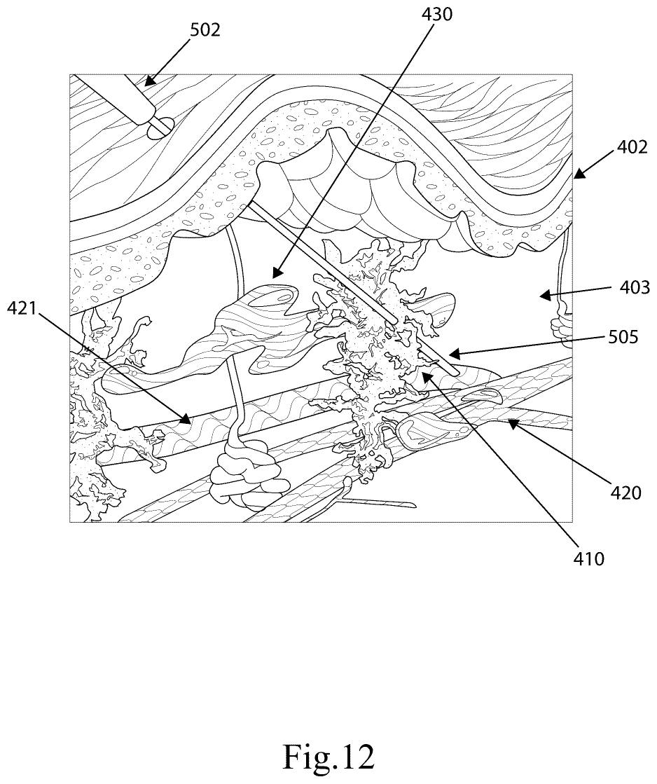

FIG. 12 is an illustration showing the blunt subcision blade/tool being used to disrupt the fibrotic tissue. Preferably, the blade 505 of blunt subcision blade 502 may be moved back and forth, subdermally, with a horizontal fan-like motion, to release the fibrous strands 410 within a part of or the entire scar area. Preferably, the blade is moved slowly with light sheering force to avoid neurovascular damage or perforation of the overlaying epidermis 402. FIG. 12 shows that the scar area is on the skin which includes epidermis 402, dermis 403, and blood vessels 420, 421. As shown, dermis 403 of the scar area has fibrotic mass 410 that is connected to epidermis 402 and the hypodermis, which causes the shown indentations and adhesions. FIG. 12 also shows that tumescent solution 430 remains in dermis 403 causing hydro-dissection.

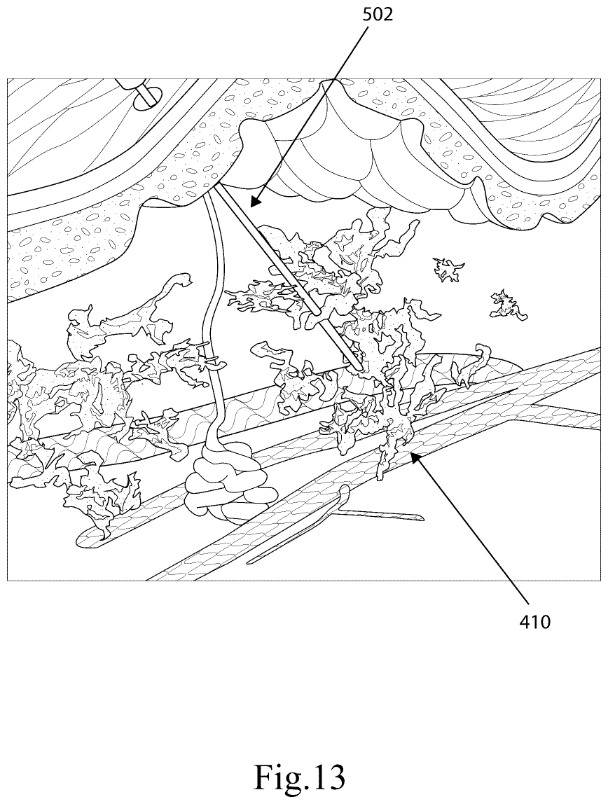

FIG. 13 is an illustration showing subcision (breaking up of fibrosis tissue) with Barikbin's blunt blade tool. FIG. 13 shows the blunt subcision blade 502 being used within the dermis to break apart fibrosis tissue 410 without damaging the skin, nerves, or blood vessels.

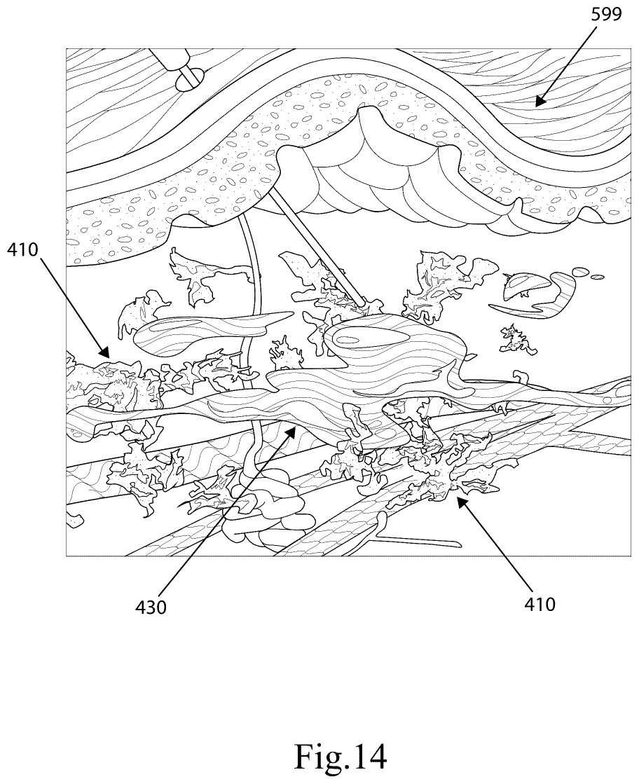

FIG. 14 is an illustration showing that the subcision step has lifted the indentations and created space within the dense fibrotic bands, preparing the area for fat injection in the following weeks, which is usually 2 weeks later. Indentations 599 are not as pronounced after the fibrotic masses 410 are broken up with Barikbin's blunt blade tool and contact with tumescent solution 430.

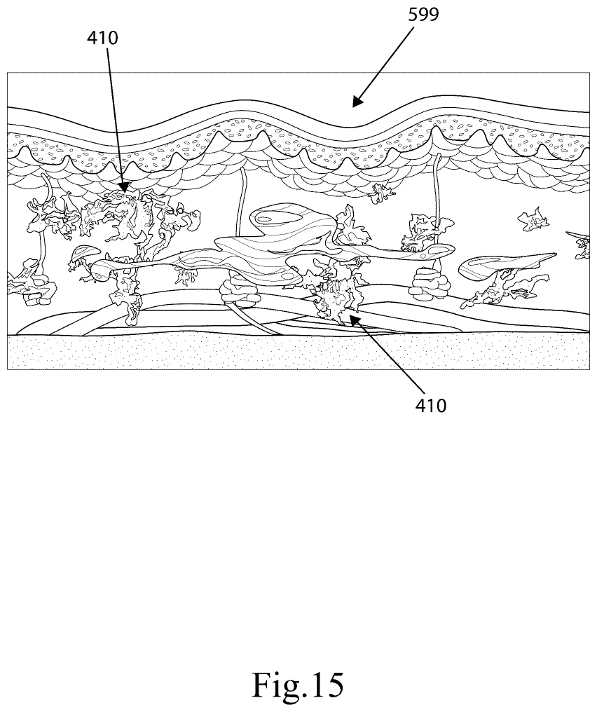

FIG. 15 is an illustration showing that the subcision step (and/or treatment with the tumescent solution) has lifted the indentations 599 and created space within the dense fibrotic bands 410, preparing the area for fat injection in the following weeks.

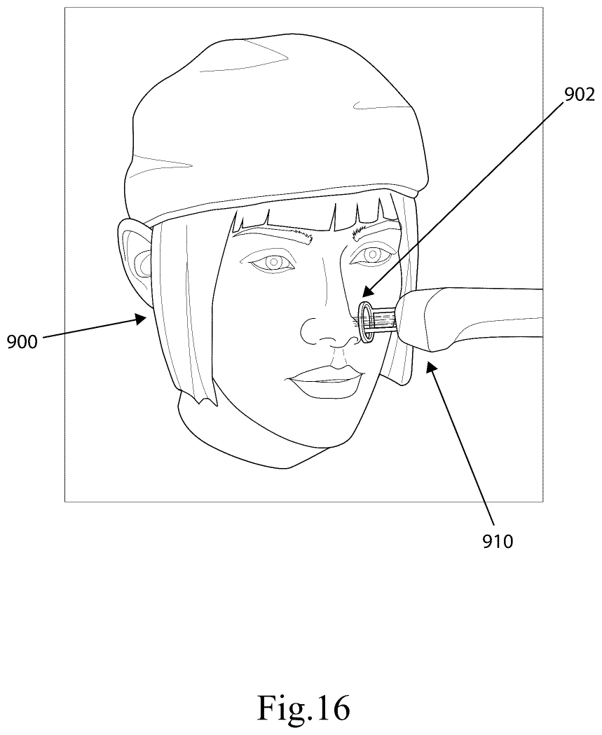

FIG. 16 is an illustration showing laser therapy. The third step of the method of the present disclosure may be laser or plasma therapy. To further enhance results, fractional CO2 laser therapy may be applied immediately after (or concurrently with) subcision. The laser therapy can be combined with, enhanced by, or replaced with, plasma-based technology, such as Plexr®. FIG. 16 shows that patient 900 has a treatment area 902 on the nose, which is treated with laser therapy machine 910.

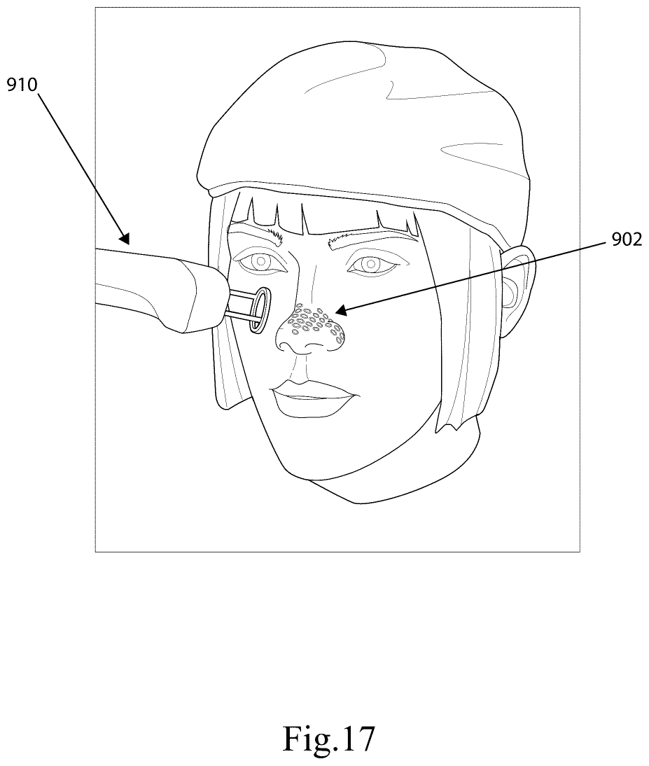

FIG. 17 is an illustration showing laser therapy may provide optimal skin rejuvenation. The method of the present disclosure seamlessly blends deeper tissue intervention (subcision) and surface level treatment (laser/plasma) achieving an artistic synergy that restores both function and aesthetic harmony. FIG. 17 shows that treatment area 902 on the nose, which is treated with laser therapy machine 910.

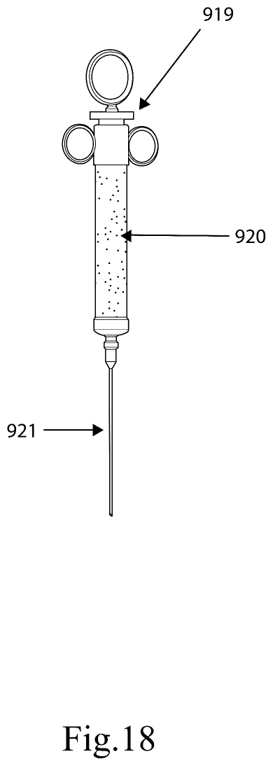

FIG. 18 is an illustration showing an injection needle with fat. About two weeks after subcision and laser therapy, as the healing process begins and swelling subsides, the positive outcomes are enhanced by fat, micro-fat, and nano-fat grafting/injection. FIG. 18 shows that the micro-fat 920, which is fat tissue that is broken down, typically via a mechanical means, into small particles, may be loaded into syringe 919 for injection into the skin via hypodermic needle 921.

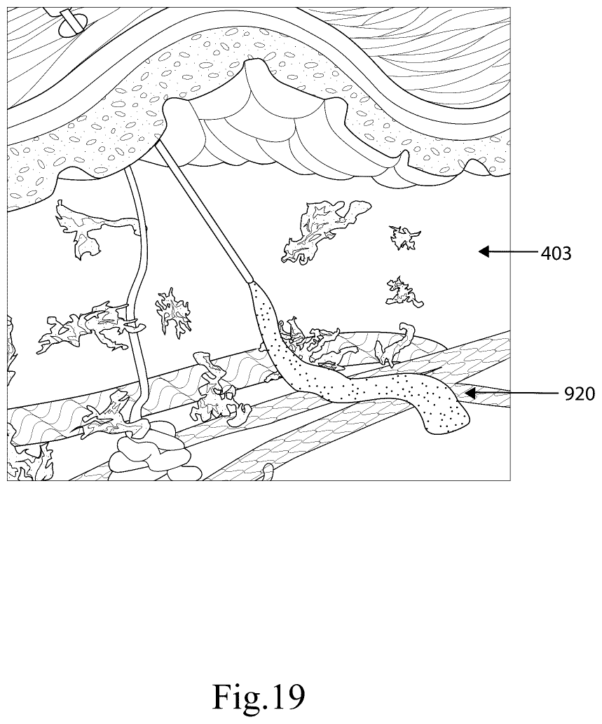

FIG. 19 is an illustration showing fat/micro-fat being injected. Step four of the method of the present disclosure may be injection of fat into the skin of the scar area. Approximately two weeks after subcision and/or laser/plasma therapy, as the healing process begins and swelling subsides, positive outcomes are enhanced by fat, micro-fat, and/or nano-fat grafting/injection. FIG. 19 shows the micro-fat 920 being injected into dermis 403. The dermis 403 no longer contains the tumescent solution and the area has healed from the subcision trauma.

Preferably, micro-fat may be autologous fat tissue harvested via gentle liposuction (commonly from the abdomen or thighs of the patient to be treated with the subcision procedure). The harvested fat may be processed through a mechanical filtration method (usually passed between syringes through 2.4 mm and 1.2 mm connectors). This preserves viable adipocytes along with components of the stromal vascular fraction (SVF), including mesenchymal stem cells and pericytes. Micro-fat is used primarily for structural support and volumization.

The particle size of the micro-fat may be approximately 400-800 microns. The injection device may be a 22G blunt-tip cannula. The typical volume used may be approximately 1-2 cc, injected beneath the scarred area in the deep dermis or hypodermis.

Nanofat may be obtained from the same lipoaspirate by further emulsifying the micro-fat through serial filtration (typically using filters down to 0.4 mm or smaller). This process removes adipocytes while preserving SVF, cytokines, and regenerative growth factors. Nanofat has a milky, fluid consistency and is used for regenerative purposes, which may include improvement in skin texture, pigmentation, vascularity, and scar remodeling.

The particle size of the nano-fat may be less than 100 microns. The injection device may be a 27G to 30G mesotherapy needle (sharp). The typical volume used may be approximately 2 cc, injected intradermally and within the scar tissue.

Together, micro-fat and nano-fat act synergistically. Micro-fat restores volume and improves blood supply. Nano-fat stimulates collagen remodeling and skin regeneration and also neovasculaization.

In this procedure, approximately 1-2 cc of micro-fat and approximately 2 cc of nano-fat may be injected approximately 2 weeks after the initial subcision and laser/plasma therapy. This timing allows for resolution of swelling and fluid absorption, providing an optimized scaffold and environment for fat grafting and cellular integration.

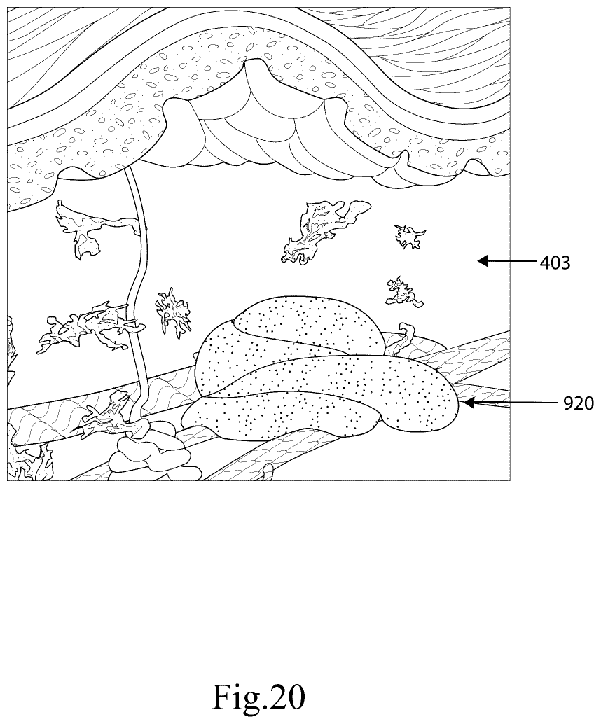

FIG. 20 is an illustration showing fat/micro fat having been injected. FIG. 20 shows that micro-fat 920 is filing dermis 403 of the scar or treatment area.

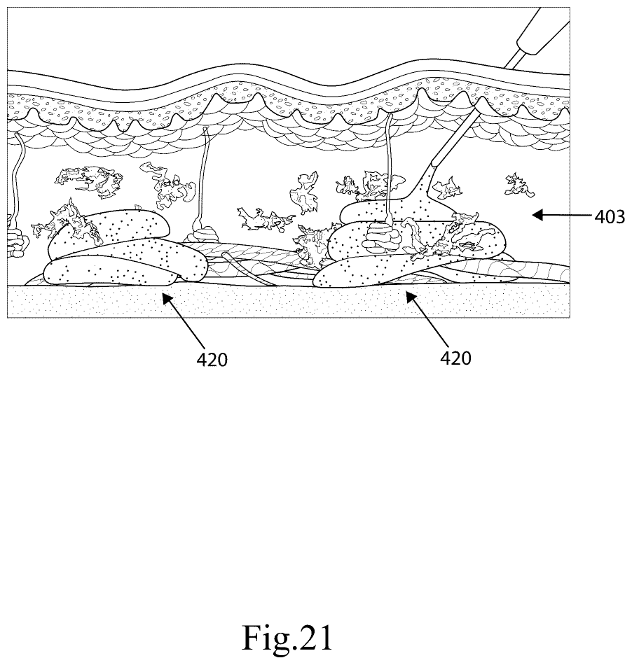

FIG. 21 is an illustration showing additional fat/micro fat being injected deep beneath the skin to fill depressed areas beneath the scar. FIG. 21 shows that micro-fat 920 may be injected into several places within dermis 403, which fills in the depressed areas of the epidermis.

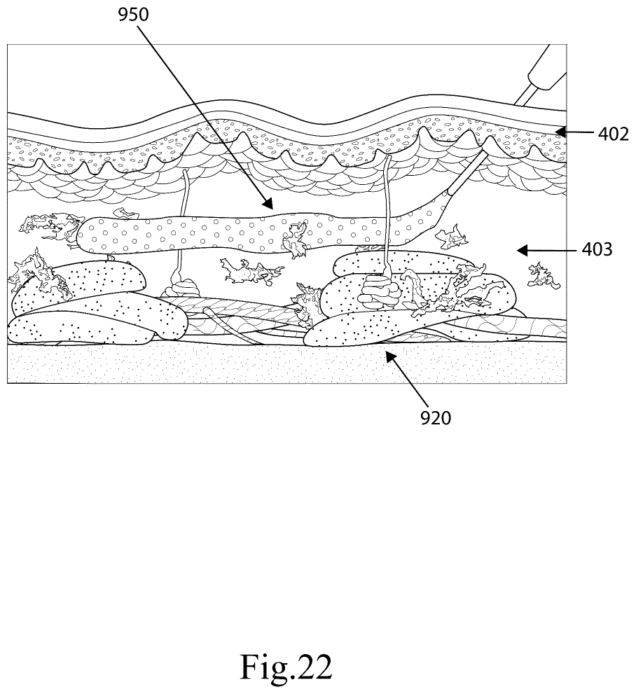

FIG. 22 is an illustration showing nano-fat is applied intradermally to rejuvenate the skin surface and refine scar texture. FIG. 22 shows that along with micro-fat 920 being injected into dermis 403, nano-fat 950 may be injected just under epidermis 402 in order to rejuvenate the skin surface and refine the scar texture.

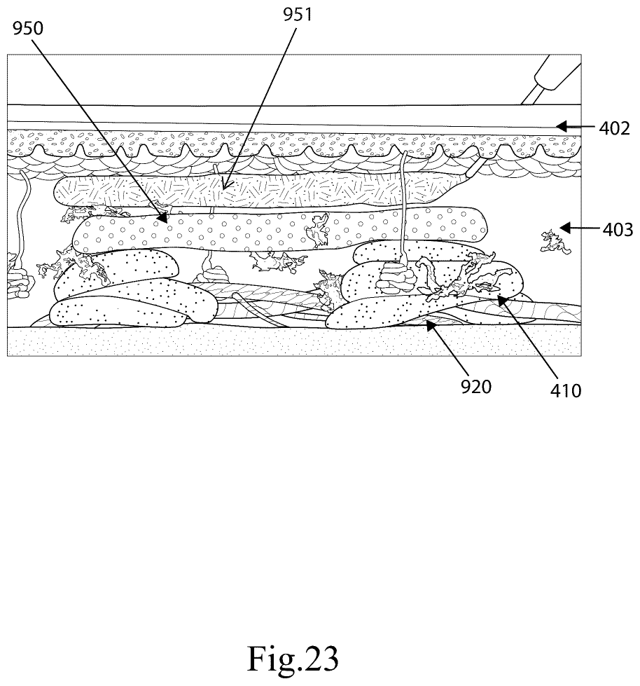

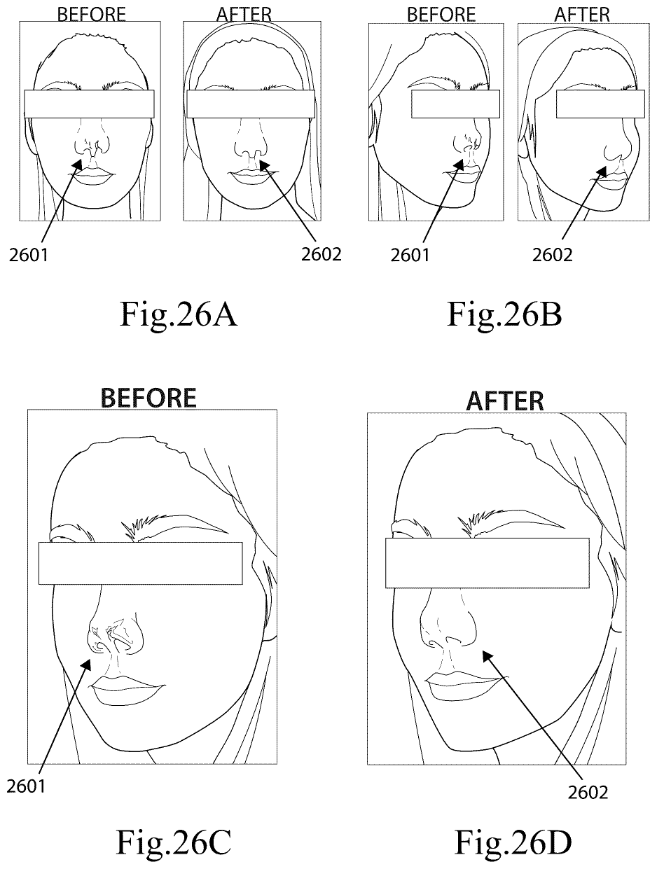

FIG. 23 is an illustration showing additional nano-fat being injected just under the top layer of skin. The method of the present disclosure may be repeated (in its entirety additional times, as needed, with some cases needing to be repeated 2-3 times) at intervals of 2-3 months depending on the patient's needs. Sometimes the results of the minimally invasive method of the present disclosure are so profound that revision surgery is no longer necessary. As shown in FIG. 26. FIG. 23 shows that along with micro-fat 920 being injected into dermis 403, nano-fat 951 may be injected just under epidermis 402 in order to rejuvenate the skin surface and refine the scar texture. Nano-fat 950 having been earlier injected. FIG. 23 shows that the epidermis 402 is substantially free of the indentations and adhesions that were causing visible scaring on the nose of the patient.

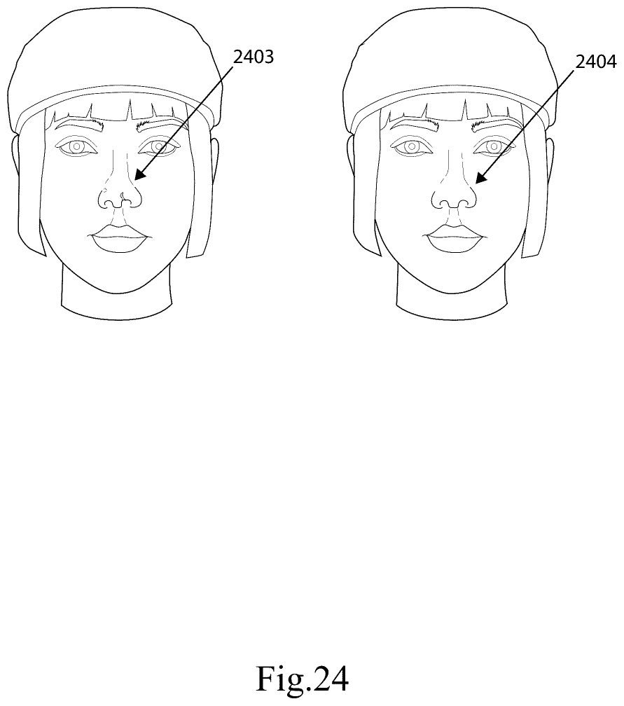

FIG. 24 is an illustration showing before and after (left and right respectively) treatment via the method of the present disclosure, which has been so successful that further revision surgery is unnecessary. In other cases, the method prepares the nasal tissue for successful revision surgery by improving skin quality, enhancing blood supply, and minimizing adhesions. FIG. 24 shows the scar area before 2403 and after 2404 the method of the present disclosure is performed. As shown, the scar area is improved dramatically after the method of the present disclosure is performed.

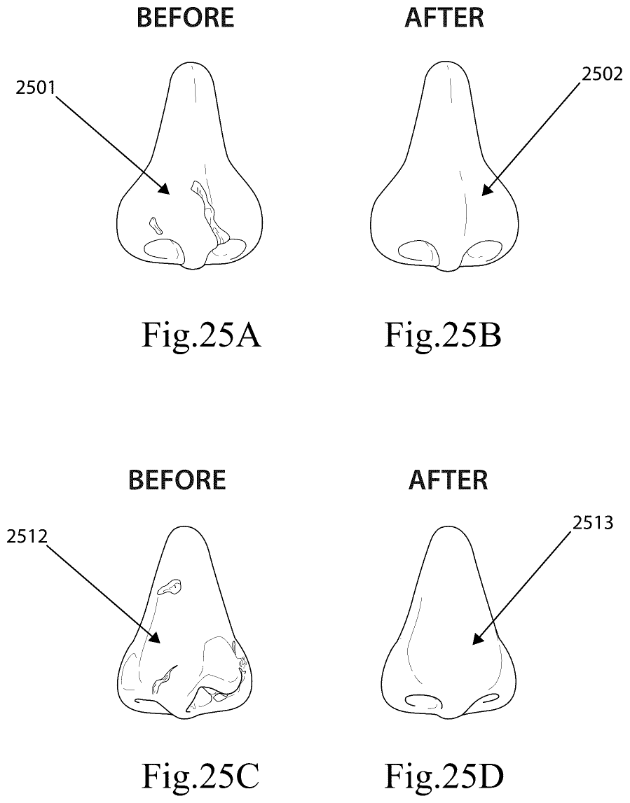

FIGS. 25A-D is an illustration of images showing positive results from the method of the present disclosure. FIGS. 25A-D show the scar area before 2501, 2512 and after 2502, 2513 the method of the present disclosure is performed. As shown, the scar area is improved dramatically after the method of the present disclosure is performed.

FIGS. 26A-D are illustrations of a patient treated with the method of the present disclosure. FIGS. 26A-D are actual photos that show the scar area before 2601 and after 2602 the method of the present disclosure is performed. As shown, the scar area is improved dramatically after the method of the present disclosure is performed.

Unless otherwise stated, all measurements, values, ratings, positions, magnitudes, sizes, locations, and other specifications that are set forth in this specification, including in the claims that follow, are approximate, not exact. They are intended to have a reasonable range that is consistent with the functions to which they relate and with what is customary in the art to which they pertain.

The foregoing description of the preferred embodiment has been presented for the purposes of illustration and description. While multiple embodiments are disclosed, still other embodiments will become apparent to those skilled in the art from the above detailed description, which shows and describes the illustrative embodiments. These embodiments are capable of modifications in various obvious aspects, all without departing from the spirit and scope of protection. Accordingly, the detailed description is to be regarded as illustrative in nature and not restrictive. Also, although not explicitly recited, one or more embodiments may be practiced in combination or conjunction with one another. Furthermore, the reference or non-reference to a particular embodiment shall not be interpreted to limit the scope of protection. It is intended that the scope not be limited by this detailed description, but by the claims and the equivalents to the claims that are appended hereto.

Except as stated immediately above, nothing that has been stated or illustrated is intended or should be interpreted to cause a dedication of any component, step, feature, object, benefit, advantage, or equivalent, to the public, regardless of whether it is or is not recited in the claims.

What is claimed is:

1. A method of treating scars comprising:
providing a tumescent solution;
injecting said tumescent solution into a scarred area of a nose of a patient;
performing subcision on said scarred area;
treating said scarred area of said nose with one or more of a laser treatment and plasma treatment; and
injecting fat into said scarred area of said nose.

2. The method of claim 1, wherein said tumescent solution comprises hyaluronic acid, lidocaine, epinephrine, and normal saline.

3. The method of claim 2, wherein between 5 to 10 cc of said tumescent solution is injected.

4. The method of claim 2, wherein said tumescent solution aids in anesthesia, reduces bleeding via vasoconstriction, and provides for hydro-dissection of said scarred area.

5. The method of claim 2, wherein said tumescent solution is 20 mL and comprises 3 mL lidocaine 2%, 0.02 mL epinephrine 1:1000, 3 mL non-crosslinked hyaluronic acid 2%, and 13.98 mL normal saline.

6. The method of claim 1, wherein said subcision is performed using a blunt subcision blade.

7. The method of claim 6, wherein a blunt blade of said blunt subcision blade is 1.5 mm to 2.2 mm in width and 50 mm to 70 mm in length, and wherein said blunt blade has a tapered non-sharp blunt tip and dual flat lateral edges.

8. The method of claim 7, wherein a plurality of fibrotic bands and adhesions are released by said blunt subcision blade without damaging nerves or blood vessels.

9. The method of claim 1, wherein said laser treatment is fractional CO2 laser treatment.

10. The method of claim 1, wherein said plasma treatment is Plexr® plasma treatment.

11. The method of claim 1, wherein said laser treatment and plasma treatment is performed immediately after said subcision step.

12. The method of claim 11, wherein said laser treatment and plasma treatment smooths an external appearance of said scar area.

13. The method of claim 1, wherein said injecting fat into said scar area step is performed approximately 2 weeks after said laser or plasma treatment step.

14. The method of claim 1, wherein said injecting fat into said scar area step comprises injecting a micro-fat and a nano-fat into said scar area.

15. The method of claim 1, wherein said injecting fat into said scar area step uses adipose stem cells, which have regenerative properties, which promotes healing of said scar area.

16. A method of treating scars comprising:
providing a tumescent solution;
injecting said tumescent solution into a scarred area of a nose of a patient;
performing subcision on said scarred area;
treating said scarred area of said nose with one or more of a laser treatment and a plasma treatment; and
injecting fat into said scarred area of said nose;
wherein said tumescent solution comprises hyaluronic acid, lidocaine, epinephrine, and normal saline;
wherein said subcision is performed using a blunt subcision blade;
wherein a plurality of fibrotic bands and adhesions are released by said blunt subcision blade without damaging nerves or blood vessels;
wherein said laser treatment and said plasma treatment is performed immediately after said subcision step;
wherein said laser treatment and said plasma treatment smooths an external appearance of said scar area;
wherein said injecting fat into said scar area step is performed approximately 2 weeks after said laser treatment and plasma treatment;
wherein said injecting fat into said scar area step comprises injecting a micro-fat and a nano-fat into said scar area; and
wherein said injecting fat into said scar area step uses adipose stem cells, which have regenerative properties, which promotes healing of said scar area.

17. A method of treating scars comprising:
identifying a scarred area on a nose of a patient;
performing subcision on said scarred area;
wherein said subcision is performed using a blunt subcision blade;
wherein a plurality of fibrotic bands and adhesions are released by said blunt subcision blade without damaging nerves or blood vessels.

18. The method of claim 17, wherein a blunt blade of said blunt subcision blade is 1.5 mm to 2.2 mm in width and 50 mm to 70 mm in length, and wherein said blunt blade has a tapered non-sharp blunt tip and dual flat lateral edges.

19. The method of claim 18, further comprising:
providing a tumescent solution;
injecting said tumescent solution into said scarred area of said nose of said patient;
treating said scarred area of said nose with one or more of a laser treatment and a plasma treatment; and
injecting fat into said scarred area of said nose.

20. The method of claim 19, wherein said tumescent solution of 20 mL comprises 3 mL lidocaine 2%, 0.02 mL epinephrine 1:1000, 3 mL non-crosslinked hyaluronic acid 2%, and 13.98 mL normal saline;
wherein a plurality of fibrotic bands and adhesions are released by said blunt subcision blade without damaging nerves or blood vessels;
wherein one or more of a laser and plasma treatment is performed immediately after said subcision step;
wherein said laser and plasma treatment step smooths an external appearance of said scar area;
wherein said injecting fat into said scar area step is performed approximately 2 weeks after said laser treatment and plasma treatment;
wherein said injecting fat into said scar area step comprises injecting a micro-fat and a nano-fat into said scar area; and
wherein said injecting fat into said scar area step uses adipose stem cells.
